(19) United States
(12) Patent Application Publication
(10) Pub. No.: US 2012/0165789 A1
(43) Pub. Date: Jun. 28, 2012

(54) Spring Action Wire Guide

(75) Inventors: Michael D. Deckard, Solsberry, IN (US); Michael W. Hardert, Bloomington, IN (US)

(73) Assignee: Cook Incorporated, Bloomington, IN (US)

(21) Appl. No.: 13/379,977

(22) PCT Filed: Jul. 8, 2010
(86) PCT No.: PCT/US10/41367

§ 371 Date: Mar. 8, 2012

Related U.S. Application Data
(60) Provisional application No. 61/224,236, filed on Jul. 9, 2009.

(51) Int. Cl. A61M 25/09

(52) U.S. Cl. 604/528

(57) ABSTRACT

A medical wire guide may include a mandrel and a cannula disposed about a portion of the mandrel. A spring may be coupled with the mandrel. The spring may be placed in a loaded state in response to relative displacement between the mandrel and the cannula. Upon release from the loaded state, the spring is configured to bias the mandrel in a distal direction relative to the cannula.

### PRIORITY CLAIM

This application claims the benefit of U.S. Provisional Application No. 61/224,236, filed Jul. 9, 2009, which is hereby incorporated by reference.

### TECHNICAL FIELD

This application relates to medical devices, and in particular to wire guides and methods of using wire guides.

### BACKGROUND

Wire guides are commonly used in vascular procedures to introduce a wide variety of medical devices into the vascular system. For example, wire guides may be used in angioplasty procedures, diagnostic and interventional procedures, percutaneous access procedures, or radiological and neuroradiological procedures in general.

A traditional wire guide may include an elongated core element with one or more tapered sections near its distal end and a flexible helical coil disposed about the distal portion of the core element. The distal end of the core element or a separate safety ribbon which is secured to the distal end of the core element may extend through the flexible coil and be secured to a distal end member of the wire guide, such as a rounded member disposed at the distal end of the helical coil. In addition, the wire guide may include a handle at the proximal end of the core element to rotate, and thereby steer the wire guide as it is being advanced through a patient's vascular system.

Wire guides may encounter various challenges as they are steered through a patient's vascular system or other body lumen. For example, a procedure may require a physician to steer a wire guide through tortuous passageways before reaching a destination. In such a procedure, the wire guide needs sufficient stiffness to be pushed along the path while remaining flexible enough to pass through the tortuous passageways without causing damage. Additionally, the patient's vascular system or other bodily lumen may contain occlusions that impede the wire guide along its path. Some wire guides may struggle to pass through occlusions. These occlusions may also impede fluid flow. Therefore, a need exists for an improved wire guide for passing through and/or clearing occlusions.

### BRIEF SUMMARY

In one implementation, a medical wire guide is provided that includes a mandrel, a cannula disposed about a portion of the mandrel, and a spring coupled with the mandrel. The spring is configured to be placed in a loaded state in response to relative displacement between the mandrel and the cannula. The spring is also configured to bias the mandrel in a distal direction relative to the cannula upon release of the spring from the loaded state.

In another implementation, a medical wire guide is provided that includes a mandrel, a cannula disposed about a first portion of the mandrel, and a spring disposed about a second portion of the mandrel. The mandrel is movable relative to the cannula to place the spring in a loaded state. The cannula comprises a first recess, and the mandrel comprises a protuberance engagable with the first recess. The spring is held in the loaded state when the protuberance is engaged with the first recess. The spring biases the mandrel forward in a distal direction relative to the cannula upon disengagement of the protuberance from the first recess.

In yet another implementation, a method of using a medical wire guide is provided. A spring of the medical wire guide is placed in a loaded state by retracting a mandrel of the medical wire guide. A distal tip of the medical wire guide, disposed at a distal end of the mandrel, is positioned to be within a spring range of an occlusion in a body lumen. The spring is released from the loaded state to bias the distal tip against the occlusion.

### BRIEF DESCRIPTION OF THE DRAWINGS

The components in the figures are not necessarily to scale. Moreover, in the figures, like referenced numerals designate corresponding parts throughout the different views.

FIG. 1 shows an embodiment of a wire guide with a spring in a substantially relaxed state.

FIG. 2 shows the wire guide of FIG. 1 with the spring in a compressed state.

FIG. 3 shows another embodiment of a wire guide that includes a spring.

FIG. 4 shows a partial perspective view of the wire guide of FIG. 3.

FIG. 5 shows another partial perspective view of the wire guide of FIG. 3.

FIG. 6 shows a partial perspective view of another embodiment of a wire guide with a spring in a substantially relaxed state.

FIG. 7 shows a partial perspective view of the wire guide of FIG. 6 with the spring in an extended state.

FIG. 8 shows another partial perspective view of the wire guide of FIG. 6.

FIG. 9 shows a method of using a spring action wire guide to pass through an occlusion.

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

As used herein, the term “proximal” refers to a portion of the wire guide (or a portion of any device component) closest to a physician when placing a wire guide in a patient, and the term “distal” refers to a portion of the wire guide (or a portion of any device component) closest to the end inserted into the patient's body.

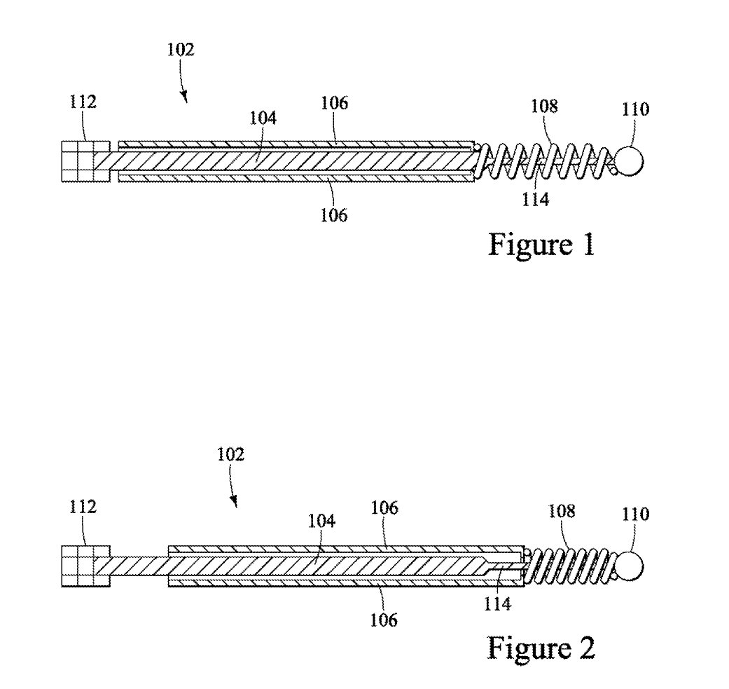

FIGS. 1 and 2 illustrate an embodiment of a wire guide 102. The wire guide 102 includes a mandrel 104, a cannula 106, a spring 108, and a distal tip 110. As described in greater detail below, the spring 108 is configured to propel the mandrel 104 and the distal tip 110 forward in a distal direction relative to the cannula 106 when the spring 108 is released from a compressed state. In some implementations, the spring 108 pushes the distal tip 110 of the wire guide 102 against an occlusion in a vascular passage or other bodily lumen with sufficient force to clear or pass through the occlusion.

The mandrel 104 may comprise a core wire or solid shaft with a distal end and a proximal end. The distal tip 110 may be disposed at the distal end of the mandrel 104. A handle 112 may be disposed at the proximal end of the mandrel 104. The handle 112 may be used to push the mandrel 104 in a distal direction, pull the mandrel 104 in a proximal direction, or twist the mandrel 104.

The mandrel 104 may be formed of a suitable metallic material such as medical grade stainless steel, a stainless steel alloy, a super-elastic material including a nickel-titanium alloy (e.g., Nitinol), a linear-elastic material, or combinations of these materials. In other implementations, other suitable mandrel materials may be used. The mandrel 104 may include a radiopaque material, such as platinum or gold. Inclusion of a radiopaque material may increase the visibility of the wire guide 102 within the body of a patient. In some implementations, a radiopaque material may be included in other portions of the wire guide 102, such as in the cannula 106, the spring 108, and/or the distal tip 110.

The mandrel 104 may take one of many different shapes. In some implementations, the mandrel 104 has a circular cross-sectional shape. In other implementations, the mandrel 104 has a rectangular cross-sectional shape. In yet other implementations, the cross-section of the mandrel 104 assumes different shapes along the length of the mandrel 104.

The mandrel 104 may have a cross-sectional area that remains substantially constant along its length. Alternatively, the mandrel 104 may have a cross-sectional area that varies along its length. In one implementation, the mandrel 104 has a cross-sectional area that diminishes gradually or stepwise at increasing distances from the proximal end of the wire guide 102 such that the mandrel 104 tapers to a smaller diameter toward its distal end. For example, as shown in FIGS. 1 and 2, the mandrel 104 may include a distal end portion 114 of a reduced diameter. The distal end portion 114 may increase the flexibility of the distal end of the wire guide 102.

The cannula 106 is disposed about a portion of the mandrel 104. The cannula 106 may be a sheath, tube, helical coil, or a combination thereof. The cannula 106 may be compressible or uncompressible. In one implementation, the cannula 106 comprises a solid tube. In another implementation, the cannula 106 comprises a coil wrapped around the mandrel 104. The cannula 106 may be manufactured from stainless steel, a stainless steel alloy, a nickel-titanium alloy (e.g., Nitinol), or combinations of these materials. In other implementations, other cannula materials may be used.

The cannula 106 is sized to receive the portion of the mandrel 104 such that the mandrel 104 is longitudinally movable relative to the cannula 106. In this way, a physician may use the handle 112 to push, pull, or twist the mandrel 104 relative to the cannula 106. In one implementation, the mandrel 104 may be coated with a material to allow it to slide through the cannula 106 more easily. In another implementation, the inner surface of the cannula 106 may be coated. The coating may be a material that reduces the coefficient of friction between the mandrel 104 and the cannula 106. For example, the coating may include a polymer, such as a fluoropolymer. In one implementation, the coating may be polytetrafluoroethylene (“PTFE”).

The distal tip 110 is disposed at a distal end of the mandrel 104. In one implementation, the distal tip 110 is an integral portion of the mandrel 104. In another implementation, the distal tip 110 is connected with the mandrel 104. For example, the distal tip 110 may be attached to the mandrel 104 by adhesive, solder, laser welding, or other attachment method. In one implementation, the distal tip 110 comprises an atraumatic shape, such as a rounded front or a front of flexible material that provides blunt force for dislodging or clearing occlusions. For example, the distal tip 110 may be a solder ball or a sphere. In another implementation, the distal tip 110 may have a shape that is designed to pierce through occlusions. For example, the distal tip 110 may be a spear, sharpened end, or other pointed structure.

The spring 108 is disposed about a distal portion of the mandrel 104 between the distal end of the cannula 106 and the distal tip 110. For example, as shown in FIG. 1, the spring 108 may be disposed about the distal end portion 114 of the mandrel 104 in a substantially relaxed state.

The spring 108 may be a compression spring formed from any material suitable for forming compression springs, such as stainless steel, alloys including stainless steel, a nickel-titanium alloy (e.g., Nitinol), or combinations of these materials. In other implementations, other spring materials may be used. For example, the spring may be a micro or miniature type spring made by the Motion Dynamics Corporation.

In one implementation, the proximal end (or other proximal portion) of the spring 108 is fixed to the distal end of cannula 106 and the distal end (or other distal portion) of the spring 108 is fixed to the distal tip 110 in a suitable manner as is known in the art, for example, by welding, soldering, or a brazed joint. Alternatively, the proximal end (or other proximal portion) of the spring 108 may be fixed to the mandrel 104. In another implementation, the ends of the spring 108 may rest against the distal end of cannula 106 and the distal tip 110. In other implementations, different suitable spring connection systems may be used.

FIG. 2 shows the wire guide 102 of FIG. 1 with the spring 108 in a compressed state. When a user pulls the mandrel 104 in a proximal direction relative to the cannula 106, the distal tip 110 retracts towards the cannula 106 against the natural bias of the spring 108 and the spring 108 is compressed between the distal tip 110 and the cannula 106. The spring 108 resists this compression load and thus the compressed state of the spring 108 is its loaded or charged state. When the user releases the mandrel 104, the spring 108 is released from the compressed state and pushes the distal tip 110 forward in the distal direction.

The spring 108 may be configured to push the distal tip 110 forward in a distal direction in a desired manner. For example, in one implementation, the spring 108 may extend up to an inch or two very quickly. In other implementations, the spring 108 may extend more than two inches very quickly. The spring 108 may be formed to have a spring constant that provides a desired biasing force according to Hooke's law, F=−kx, where F represents the force exerted by the spring 108 when released, k represents the spring constant, and x represents the distance that the spring 108 is compressed from a relaxed state. To increase the force provided by the spring 108, the spring 108 may be compressed to a greater degree before release or may be formed to have a larger spring constant. In some implementations, a large spring force may be desired to help clear or pass through occlusions in the vascular system. In other implementations, a smaller spring force may be desired to avoid damage to the vascular system. The spring compression degree and spring constant may therefore be selected or varied to meet the needs of the intended application for the wire guide 102.

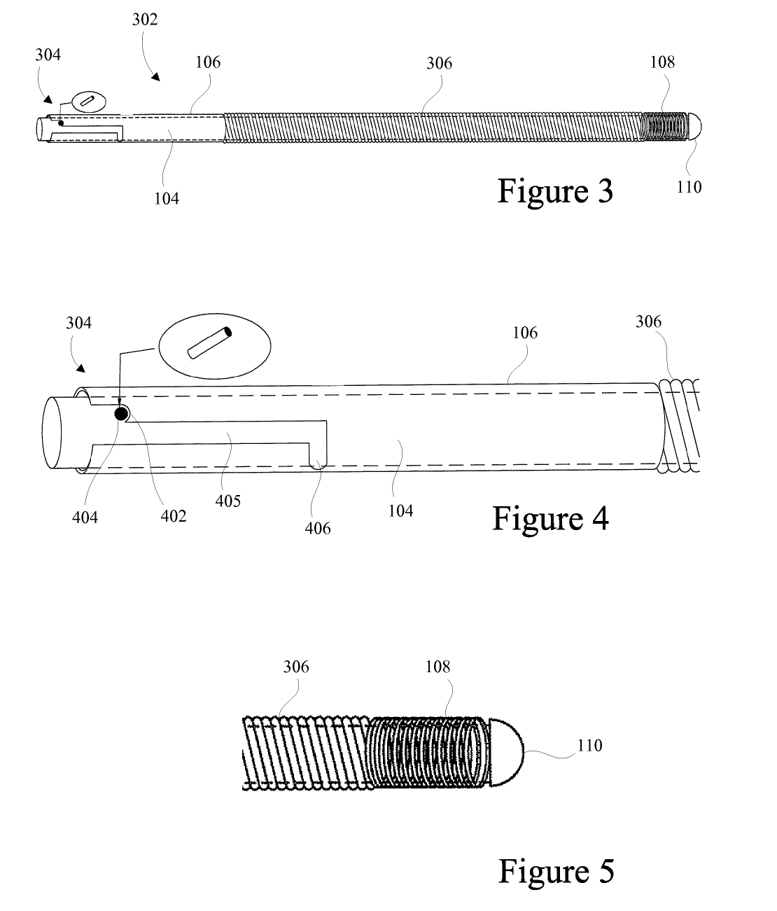

FIG. 3 shows another embodiment of a wire guide 302 that includes a mandrel 104, a spring 108, and a distal tip 110. The wire guide 302 also includes a trigger mechanism 304, which is described below in connection with FIG. 4, and a cannula 106 with at least one coil portion 306, which is described below in connection with FIG. 5.

FIG. 4 shows a partial perspective view of a proximal end portion of the wire guide 302 of FIG. 3. The trigger mechanism 304 is configured to hold the spring 108 in its compressed state and selectively release the spring 108 from the compressed state to bias the distal tip 110 forward in a distal direction.

In one implementation, the trigger mechanism 304 includes a first recess 402 located on the cannula 106 at a predetermined location near the proximal end of the cannula 106. The first recess 402 is configured to receive and engage a protuberance 404 on the mandrel 104 so as to hold the spring 108 in its compressed state. The trigger mechanism 304 also includes a longitudinal slot 405 located on the cannula 106, the proximal end of the longitudinal slot 405 being adjacent the first recess 402. The longitudinal slot 405 is configured to guide the protuberance 404 toward the first recess 402 when a user pulls the mandrel 104 in a proximal direction to compress the spring 108 from its substantially relaxed state. In operation, a user pulls the mandrel 104 in a proximal direction to compress the spring 108 until the protuberance 404 reaches the first recess 402, at which time the user twists the mandrel 104 so as to position the protuberance 404 in the first recess 402. Subsequently, when the user releases the mandrel 104, the protuberance 404 is engaged within first recess 402 under the bias of the spring 108 so as to hold the spring 108 in its compressed state. As shown in FIG. 4, the first recess 402 may include at least one concave inner surface to more reliably engage the protuberance 404 and prevent accidental dislodgment.

The location of the recess 402 may be selected to achieve the desired degree of compression when the spring 108 is in the compressed or loaded state. In implementations that desire a high degree of spring compression, the recess 402 may be located relatively close to the proximal end of the wire guide 302. In implementations that desire a lesser degree of spring compression, the recess 402 may be located relatively further from the proximal end of the wire guide 302. The trigger mechanism 304 may also include additional recesses similar to the recess 402 to allow for a user-selected amount of spring compression.

In some implementations, the trigger mechanism 304 may also include a second recess 406 located on the cannula 106 at a predetermined location adjacent the distal end of the longitudinal slot to hold the spring in a substantially relaxed state when the protuberance 404 engages the second recess 406. In other implementations, the protuberance that engages with the second recess 406 may be different than the protuberance 404 that engages with the first recess 402. The location of the second recess 406 may be selected to achieve the desired degree of flexibility in the spring 108 in a substantially relaxed state. In implementations that desire a relatively high degree of wire guide tip flexibility, the second recess 406 may be located at a position that holds the spring 108 in a relaxed state to provide a highly flexible wire guide tip. In implementations that desire a lesser degree of wire guide tip flexibility, the second recess 406 may be located at a position that partially compresses the spring 108 to provide a stiffer wire guide tip. Similarly, in implementations that desire a stiff wire guide tip, the user may engage the protuberance 404 with the first recess 402 to place the spring in the compressed state. The spring 108 provides a greater stiffness in the compressed state than it does in a substantially relaxed state. Therefore, the wire guide 302 may provide a variable range of distal tip flexibility based on the degree of compression in the spring 108.

FIG. 5 shows a partial perspective view of a distal end portion of the wire guide 302 of FIG. 3. The coil portion 306 of the cannula 106 comprises a plurality of windings around the mandrel 104 to provide the desired flexibility, pushability and torquability characteristics for the wire guide 302. The coil portion 306 may be formed of a helical wound ribbon-shaped or round wire. The windings of coil portion 360 may be closely spaced so that adjacent windings touch each other, as shown in FIG. 5. Alternatively, the windings of coil portion 360 may be spaced apart. The coils of the spring 108 may be spaced further apart in a relaxed state than the windings of the coil portion 306 in a relaxed state so that when the mandrel 104 is moved in a proximal direction relative to the cannula 106, the coils of the spring 108 compress to a greater degree than the windings of the coil portion 306.

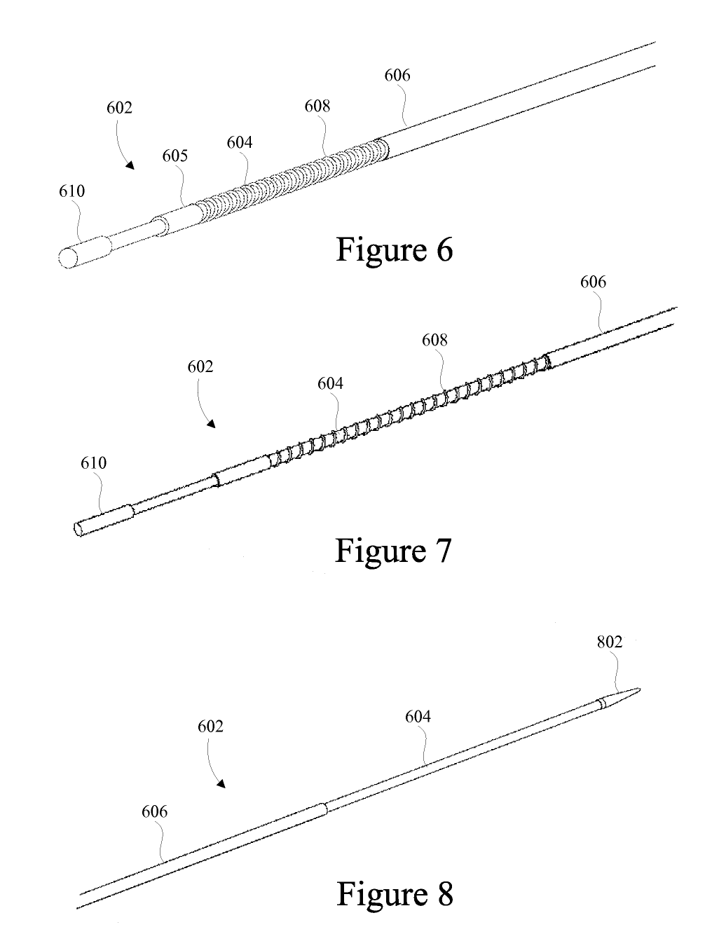

FIGS. 6-8 show another embodiment of a wire guide 602. The wire guide 602 may include a mandrel 604, a cannula 606, a spring 608, a handle 610, and a distal tip 802. The various components of the wire guide 602 may have similar features, materials, or operation as the corresponding components of the wire guides 102 and 302 described above in connection with FIGS. 1-5.

In the wire guide 602, the spring 608 is disposed about a proximal portion of the mandrel 604. Placing the spring 608 near the proximal end of the wire guide 602 allows the spring 608 to be nearer to the user and may provide the user with improved control. The spring 608 may be a tension spring formed from any material suitable for forming tension springs, such as stainless steel, alloys including stainless steel, a nickel-titanium alloy (e.g., Nitinol), or combinations of these materials. In other implementations, other elastic spring materials may be used. FIG. 6 shows the spring 608 in a substantially relaxed state.

The spring 608 may be coupled between a collar 605 mounted on the proximal portion of the mandrel 604 and the proximal end of the cannula 606. In one implementation, the proximal end (or other proximal portion) of the spring 608 is fixed to the collar 605 and the distal end (or other distal portion) of the spring 608 is fixed to the proximal end of cannula 106 in a suitable manner as is known in the art, for example, by welding, soldering, or a brazed joint. In other implementations, another spring connection system may be used. Alternatively, the proximal end (or other proximal portion) of the spring 608 may be fixed to the handle 610.

FIG. 7 shows a partial perspective view of the wire guide 602 with the spring 608 in an extended state. When a user pulls the mandrel 604 in a proximal direction relative to the cannula 606, the proximal end of the spring 608 is stretched in proximal direction with the mandrel 604 while the distal end of the spring 608 is held in place with the cannula 606. The spring 608 resists this tension load and thus the extended state is its loaded or charged state. When the user releases the mandrel 604, the spring 608 is released from the extended state and pushes the mandrel 604 forward in the distal direction relative to the cannula 606.

The spring 608 may be formed to have a spring constant that provides a desired biasing force according to Hooke's law, F=−kx, where F represents the force exerted by the spring 608 when released, k represents the spring constant, and x represents the distance that the spring 608 is stretched from a relaxed state. To increase the force provided by the spring 608, the spring 608 may be stretched to a greater degree before release or may be formed to have a larger spring constant. In some implementations, a large spring force is desired to help clear or pass through occlusions in the vascular system. In other implementations, a smaller spring force may be desired to avoid damage to the vascular system. The spring extension degree and spring constant may therefore be selected or varied to meet the needs of the intended application for the wire guide 602.

Although not shown in FIGS. 6-8, the wire guide 602 may also include a trigger mechanism, similar to the one described in connection with FIG. 4, for holding the spring 608 in an extended state and selectively releasing the spring 608 from the extended state to propel the distal tip 802 forward in a distal direction. For example, a trigger mechanism of the wire guide 602 may include a recess and a slot located on the cannula 606 at a predetermined location near the distal end of the cannula configured to receive and engage a protuberance on the mandrel 604 so as to hold the spring 608 in its extended state. When a user pulls the mandrel 604 in a proximal direction to stretch the spring 608, the protuberance travels in the slot and is engaged within the recess so as to hold the spring 608 in its extended state.

FIG. 8 shows the distal end portion of the wire guide 602. When the mandrel 604 is pulled proximally relative to the cannula 606, the distal tip 802 is pulled in the proximal direction towards the distal end of the cannula 606. After the mandrel 604 is released from this retracted state, the spring 608 (FIG. 7) can propel the mandrel 604 and the distal tip 802 forward in a distal direction against an occlusion in a vascular passage or other bodily lumen with sufficient force to clear or pass through the occlusion. The distal tip 802 may comprise an atraumatic shape or may have a shape that is designed to pierce through occlusions. For example, FIG. 8 shows the distal tip 802 with a pointed or sharpened end.

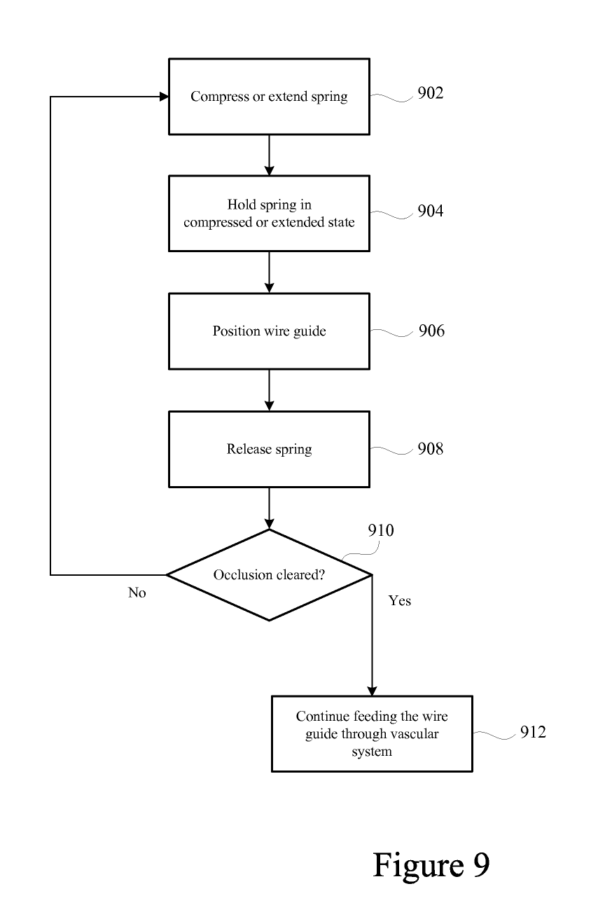

FIG. 9 shows a method of using a spring action wire guide to pass through an occlusion. An occlusion may be a partial or total blockage in a vascular passage or other bodily lumen. The method of FIG. 9 will be described with reference to the wire guide 302 shown in FIGS. 3-5 and the wire guide 602 shown in FIGS. 6-8. However, the method of FIG. 9 may also be performed with other wire guides, such as the wire guide 102 shown in FIGS. 1 and 2.

At act 902, a spring of the wire guide is compressed or stretched. The spring of the wire guide 302 or 602 may be compressed or stretched either before insertion of the device into the patient's vascular system or after insertion of the device into the patient's vascular system. In one implementation, a user may pull the mandrel 104 of the wire guide 302 in a proximal direction to retract the distal tip 110 and place the spring 108 into a compressed state. The spring 108 may be compressed between the distal tip 110 and some other support structure, such as the cannula 106 of the wire guide 302. In another implementation, a user may pull the mandrel 604 or handle 610 of the wire guide 602 in a proximal direction to place the spring 608 into an extended state. The amount of force that is provided by the spring is dependent on the amount of compression or extension in the spring. The user may control the amount of spring force created by controlling the amount of compression/extension provided to the spring. In one implementation, a small spring force may be desired. Therefore, the user may only compress/stretch the spring a relatively small amount. In other implementations, a larger spring force may be desired. Therefore, the user may compress/stretch the spring a relatively larger amount.

At act 904, the spring is held in the compressed or extended state. In one implementation, a user may engage the mandrel 104 with a trigger mechanism 304 to hold the spring 108 in the compressed state. In another example, a user may engage the mandrel 604 with a trigger mechanism to hold the spring 608 in the extended state.

At act 906, the wire guide 302 or 602 is positioned within a patient's vascular system. The wire guide 302 or 602 may be positioned within a patient's vascular system at act 906 before and/or after the spring of the wire guide 302 or 602 is placed in the loaded state at act 902. For example, in one implementation, the user may first position the distal tip of the wire guide to be near the occlusion before placing the spring in the loaded state. In another implementation, the spring may be placed in the loaded state before the distal tip is positioned to be near the occlusion.

In one implementation, a user may position the distal tip 110 of the wire guide 302 to be within a spring extension range of an occlusion in a vascular passage. The spring extension range may be the distance that the spring 108 may propel the distal tip 110 forward in a distal direction when the spring 108 is released from the compressed state. In another implementation, a user may position the distal tip 802 of the wire guide 602 to be within a spring recoil range of an occlusion in a vascular passage. The spring recoil range may be the distance that the spring 608 may propel the distal tip 802 forward in a distal direction when the spring 608 is released from the extended state.

At act 908, the spring of the wire guide is released from the compressed or extended state. In one implementation, the spring 108 is released from the compressed state. In another implementation, the spring 608 is released from the extended state. When released from the compressed or extended state, the spring propels the distal tip forward in a distal direction. The spring may be configured to extend from the compressed state quickly. The quick movement of the spring may allow the distal tip to penetrate the occlusion. Because the distal tip may be positioned to be within the spring extension or recoil range of the occlusion at act 906, the distal tip may make contact with the occlusion when propelled by the spring. In some instances, the spring 108 will push the distal tip against the occlusion without passing through the occlusion. In other instances, the spring will push the distal tip through the occlusion. A user may also twist the mandrel connected with the distal tip to apply a drilling motion from the distal tip to the occlusion.

At act 910, it is determined whether the occlusion has been sufficiently cleared. The user may determine whether a large enough passage has been created by the spring action of the wire guide. If the user determines that the occlusion has not been sufficiently cleared at act 910, then the spring may be recompressed/stretched at act 902 for a second attempt at clearing the occlusion. This recompression/stretching may occur while the distal tip remains within the patient. After one or more compressions/stretches and releases of the spring, the user may determine that the occlusion is sufficiently cleared. When that occurs, the user may continue feeding the wire guide through the patient's vascular system to the desired destination at act 912. In some instances, the objective of the procedure may be to clear one or more occlusions. In that case, the wire guide 302 would be steered to the next occlusion and the spring action occlusion clearing process may begin again for the next occlusion.

At various points in the procedure, the user may slide other medical devices over the wire guide into the patient. In one implementation, the user may slide tubing along the wire guide to the location of the occlusion. The tubing may then be used as a passageway to remove material that may become dislodged from the occlusion by the spring action wire guide. Alternatively, the tubing may be used to support a weak vessel or deliver material to a desired location within the patient.

Although the invention has been described and illustrated with reference to specific illustrative embodiments thereof, it is not intended that the invention be limited to those illustrative embodiments. For example, FIGS. 1-5 illustrate a distal end spring that is compressed and released to perform the spring action. Other alternative embodiments could include a distal end spring that is stretched and then released. Similarly, FIGS. 6-8 illustrate a proximal end spring that is stretched and released to perform the spring action. Other alternative embodiments could include a proximal end spring that is compressed and then released. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

The medical devices described herein may be dimensioned to fit within a vascular passage or other body lumen. The wire guide may generally have a length in the range of 30-600 cm. In some implementations, the length of the wire guide may be in the range of 90-300 cm. The wire guide may generally have an outer diameter in the range of 0.204-1.321 mm (0.008-0.052 inches). In some implementations, the outer diameter may be in the range of 0.254-2.286 mm (0.01-0.09 inches). For example, one type of wire guide may have an outer diameter of about 0.889 mm (0.035 inches).

What is claimed is:

1. A medical wire guide, comprising:
a mandrel;
a cannula slidably receiving a portion of the mandrel therein; and
a spring coupled with the mandrel, wherein the spring is configured to be placed in a loaded state in response to relative displacement between the mandrel and the cannula, and wherein the spring is configured to bias the mandrel in a distal direction relative to the cannula upon release of the spring from the loaded state.

2. The medical wire guide of claim 1, wherein a distal portion of the spring is coupled with the cannula, wherein a proximal portion of the spring is coupled with the mandrel, and wherein the spring is a tension spring that is extended in the loaded state.

3. The medical wire guide of claim 1, wherein a distal portion of the spring is coupled with a distal end member of the mandrel, wherein a proximal portion of the spring is coupled with the cannula, and wherein the spring is a compression spring that is compressed in the loaded state.

4. The medical wire guide of claim 3, wherein at least a portion of the cannula comprises a coil of windings disposed about a portion of the mandrel, and wherein the spring is configured to compress to a greater degree than the coil of windings in response to the relative displacement.

5. The medical wire guide of claim 1, further comprising a trigger mechanism configured to selectively engage the mandrel to hold the spring in the loaded state.

6. The medical wire guide of claim 5, wherein the trigger mechanism comprises a first recess located on the cannula, and wherein the mandrel comprises a protuberance configured to engage with the first recess to hold the spring in the loaded state.

7. The medical wire guide of claim 6, wherein the trigger mechanism comprises a longitudinal slot located on the cannula adjacent to the first recess, the longitudinal slot receiving the protuberance of the mandrel therein to guide movement of the mandrel relative to the cannula, and wherein the longitudinal slot comprises a distal end portion configured to stop further distal movement of the mandrel relative to the cannula when the protuberance makes contact with the distal end portion of the longitudinal slot.

8. The medical wire guide of claim 7, wherein the trigger mechanism comprises a second recess located on the cannula, wherein the longitudinal slot is disposed between the first recess and the second recess, and wherein the second recess is configured to hold the spring in a substantially relaxed state when the protuberance of the mandrel is engaged with the second recess.

9. The medical wire guide of claim 1, further comprising a distal tip disposed at a distal end of the mandrel, and wherein the distal tip is a distal-most portion of the medical wire guide, and wherein the distal tip moves with the mandrel when the spring biases the mandrel upon release of the spring from the loaded state.

10. The medical wire guide of claim 9, wherein the distal tip comprises a spear, sharpened end, or pointed structure at a leading end of the distal tip.

11. A medical wire guide, comprising:
a mandrel;
a cannula disposed about a first portion of the mandrel;
a spring disposed about a second portion of the mandrel, wherein the mandrel is movable relative to the cannula to place the spring in a loaded state;
wherein the cannula comprises a first recess, wherein the mandrel comprises a protuberance engagable with the first recess, wherein the spring is held in the loaded state when the protuberance is engaged with the first recess, and wherein the spring biases the mandrel forward in a distal direction relative to the cannula upon disengagement of the protuberance from the first recess.

12. The medical wire guide of claim 11, wherein the cannula further comprises a second recess and a slot extending between the first recess and the second recess, wherein the protuberance is slidably engaged with the slot when the protuberance is positioned between the first recess and the second recess, and wherein the spring is held in a substantially relaxed state when the protuberance is engaged with the second recess.

13. A method of using a medical wire guide, comprising:
placing a spring of the medical wire guide in a loaded state by retracting a mandrel of the medical wire guide;
positioning a distal tip of the medical wire guide, disposed at a distal end of the mandrel, to be within a spring range of an occlusion in a body lumen; and
releasing the spring from the loaded state to bias the distal tip against the occlusion.

14. The method of claim 13, wherein the loaded state comprises an extended state, and wherein the step of placing the spring in the loaded state comprises pulling a mandrel connected with the spring in a proximal direction to stretch the spring into the extended state.

15. The method of claim 13, wherein the loaded state comprises a compressed state, and wherein the step of placing the spring in the loaded state comprises pulling a mandrel connected with the distal tip in a proximal direction to compress the spring into the compressed state.

16. The method of claim 13, wherein the medical wire guide comprises a mandrel and a cannula disposed about a portion of the mandrel, wherein the mandrel is moveable relative to the cannula, wherein a portion of the spring is connected with the mandrel, and wherein the method further comprises:
coupling the mandrel with a trigger mechanism to hold the spring in the loaded state; and
uncoupling the mandrel from the trigger mechanism to release the spring from the loaded state to bias the distal tip against the occlusion.

17. The method of claim 13, wherein the medical wire guide comprises a mandrel and a cannula disposed about a portion of the mandrel, wherein the mandrel is moveable relative to the cannula, wherein a portion of the spring is connected with the mandrel, and wherein the method further comprises:
engaging a protuberance of the mandrel with a first recess in the cannula to hold the spring in the loaded state; and
disengaging the protuberance of the mandrel from the first recess to release the spring from the loaded state to bias the distal tip against the occlusion.

18. The method of claim 17, further comprising engaging the protuberance of the mandrel with a second recess in the cannula to hold the spring in a substantially relaxed state.

19. The method of claim 13, wherein the step of releasing comprises releasing the spring for a first attempt at propelling the distal tip through the occlusion, and wherein the method further comprises:
pulling a mandrel connected with the spring in a proximal direction to compress or stretch the spring while the distal tip is within the body lumen to return the spring to the loaded state; and
releasing the spring from the loaded state for a second attempt at propelling the distal tip through the occlusion.

20. The method of claim 13, further comprising:
inserting a proximal end of the medical wire guide though a passage in a second medical device; and
sliding the second medical device over the medical wire guide into the body lumen and towards a distal end of the medical wire guide.
